(19) United States
(12) Patent Application Publication
(10) Pub. No.: US 2026/0365784 A1
(43) Pub. Date: Nov. 19, 2026

(54) OLIGOMERIZATION OF ALPHA HYDROXY ACIDS

(71) Applicant: PRAJ INDUSTRIES LIMITED, Pune (IN)

(72) Inventors: Sayali Prafulla Jadhav, Pune (IN); Yogesh Ramesh Nevare, Pune (IN); Mangesh Ganesh Kulkarni, Pune (IN); Pramod Shankar Kumbhar, Pune (IN); Jayant Shridhar Sawant, Pune (IN)

(21) Appl. No.: 19/132,089

(22) PCT Filed: Nov. 9, 2023
(86) PCT No.: PCT/IN2023/051036

§ 371 Date: May 22, 2025

(30) Foreign Application Priority Data
Nov. 23, 2022 (IN) 202221067248

(51) Int. Cl. C08G 63/08; B01J 3/00; B01J 19/00; C08G 63/78

(52) U.S. Cl. CPC C08G 63/08; B01J 3/006; B01J 19/0013; B01J 19/0066; C08G 63/785

(57) ABSTRACT

The present disclosure relates to a system (100) assembled for carrying out oligomerization of α-hydroxy acids comprising: a reaction vessel (101), distillation column (102), an inlet valve (103), reflux divider (104), condenser (105) a vessel (108) containing α-hydroxy acids for feeding, peristaltic pump (109), stirring means (110) for stirring the reaction vessel (101), heating means (111) for heating the reaction vessel (101), means (112) to measure temperature of the reaction vessel (101), and means (113) for heating, adjusting, regulating or maintaining temperature of the distillation column (102), characterized in that, the α-hydroxy acids are fed portion-wise to the reaction vessel (101) via the inlet valve (103) positioned close to the distal end of the distillation column (102) in a controlled mode; such that oligomers of MW ranging from 900-1200 Da are synthesized/obtained within 5 hours, with less than 2% of α-hydroxy acids in the distillate collector (107).

### CROSS-REFERENCE TO RELATED APPLICATIONS AND PRIORITY

The present application claims priority from Indian patent application number (202221067248) filed on (23 Nov. 2022), incorporated herein by a reference.

### TECHNICAL FIELD

The present subject matter, in general, relates to a method for synthesizing oligomers. More particularly, the present subject matter relates to a system assembled for carrying out oligomerization of α-hydroxy acids.

### BACKGROUND

α-Hydroxy acids, or alpha hydroxy acids (AHAs), are a class of naturally occurring chemical compounds that consist of a carboxylic acid substituted with a hydroxyl group on the adjacent carbon. While α-hydroxy acids are classically prepared by the addition of hydrogen cyanide to a ketone or aldehyde, followed by acidic hydrolysis of the resulting cyanohydrin product, they are also routinely produced by fermenting chemical components under controlled conditions, for instance, treating an aldehyde and prussic acid or α-hydroxynitriles with a microorganism having nitrilase or nitrile hydratase activity in an aqueous medium. Prominent examples of AHAs are glycolic acid, lactic acid, and citric acid.

The polymerization products of α-hydroxy acids are an interesting class of biodegradable, biocompatible, bioresorbable polymers that decompose into physiologically tolerable, non-toxic degradation products that are eliminated from the organism or completely bio absorbed. Other advantages of poly (α-hydroxy acid) utilization in biomedical applications are, possibility of controlling their physio-mechanical properties, chemical or physical modification of their surface properties, ability to immobilize cells or biomolecules within them or on the surface. For this reason, they have found interesting applications in the last decade in technologies such as, tissue engineering, skeletal systems, cardiovascular devices, artificial organs, ophthalmology, controlled drug delivery systems, etc. These biodegradable polymers do not evoke a sustained inflammatory or toxic response upon implantation in the body, and their degradation time can match the healing or regeneration process, thus demonstrating appropriate permeability and processability for the intended biomedical application. Poly (α-hydroxy acids), especially poly (glycolic acid) (PGA), poly(lactic acid) (PLA) and their copolymers poly (lactic-co-glycolic acid) (PLGA) are novel class of commodity polymers, used extensively in biomedical applications.

It is conventionally known that the synthesis of poly (lactic acid), and poly(glycolic acid) involve four major steps; i) oligomerization of α-hydroxy acids to desired molecular weight (degree of polymerization (DP)=8-45) ii) depolymerization of oligomers and formation of cyclic dimers iii) purification of cyclic dimers and iv) polymerization by ring opening of the dimers.

Ring Opening Polymerization (ROP) of cyclic diesters yields higher molecular weight polymers (MW>100,000 g/mol) in a relatively short time; favouring this mechanism for industrial mass production. The polymers produced in the ROP reaction are typically linear and have a narrow range of molecular weight distribution, which is normally difficult to achieve by other polymerization techniques. This process is particularly useful in the polymerization of α-hydroxy acids such as lactic acid that defines the yield, and desired properties of the final polymer product. The condensation of α-hydroxy acids to its oligomers conventionally takes place by the removal of water by-product, which can advantageously be achieved by simple evaporation, in one or a plurality of steps, at atmospheric pressure or under reduced pressure, until an oligomeric composition is obtained having an average DP ranging from 8-45. Oligomerization of α-hydroxy acids may also be performed using solvents that form azeotropes with water for removing the water formed during the condensation reaction; mixing and boiling the mixture; stabilizing the amount of solvent in reaction medium and then distilling out the remaining/extra water.

Substantial amounts of α-hydroxy acids such as, glycolic acid and lactic acid are usually lost during the oligomerization process as they co-distill with water. This takes place due to the high affinity of α-hydroxy acids to water, and especially when the boiling points of α-hydroxy acids are close to the range of boiling point of water.

This makes the process of water removal by distillation during oligomerization extremely difficult. Furthermore, formation of oligomers by carrying out conventional water distillation requires long reaction times. As a result, many a times, the obtained oligomers do not possess the desired properties.

Prior art describes oligomerization carried out in a batch reactor, such that the time required is >6 hrs. In addition to the long duration of the reaction, the processes cited in art are complicated and involve the presence of complex structural or procedural elements such as stirred tank reactor, rectifying column having multiple sections, falling film reboiler, homogeneous catalyst, water removal at high vacuum such as 100-200 mbar, etc.

However, prior art does not teach continual feeding of lactic acid in a fractional distillation column, that reduces loss of lactic acid and decreases the overall time required for carrying out oligomerization.

In pursuance of this, the inventors of the present disclosure were motivated to congregate an oligomerization assembly to carry out rapid oligomerization of α-hydroxy acids by feeding α-hydroxy acids in portions or continually through a packed column and using the same column to avoid the loss of α-hydroxy acids along with water.

### SUMMARY

The present invention is directed to a system 100 assembled for carrying out oligomerization of a α-hydroxy acids, the said system assembly 100 comprising: a reaction vessel 101 charged with a portion of the total α-hydroxy acids configured to connect to proximal end of a distillation column 102; the distillation column 102 configured to receive α-hydroxy acids via an inlet valve 103 close to its distal end; the inlet valve 103 for feeding the remaining α-hydroxy acids in a controlled manner configured to connect to a vessel 108; the distal end of the distillation column 102 further configured to connect to proximal end of a reflux divider 104; the distal end of the reflux divider 104 configured to connect to proximal end of a condenser 105, while being provided with means for connecting to the distillate collector 107; distal end of the condenser 105 and the distillate collector 107 being provided with means for connecting to a vacuum pump 106; characterized in that, the α-hydroxy acids are fed portion-wise or continually to the reaction vessel 101 via the inlet valve 103. In a related exemplary embodiment, the instant invention discloses a process for carrying out oligomerization of α-hydroxy acids by the system 100 vide supra; said process comprising: (a) charging reaction vessel 101 with a portion of the total α-hydroxy acids; (b) reducing and further maintaining reduced pressure of said system 100; (c) heating reaction vessel 101 to reflux temperature to enable oligomerisation of the α-hydroxy acids; (d) maintaining temperature of the distillation column 102 to enable evaporation of water; (e) feeding the remaining α-hydroxy acids to the reaction vessel 101 via inlet valve 103 for further oligomerization; (f) maintaining the reflux temperature of reaction vessel 101 to effect oligomerization of α-hydroxy acids with simultaneous removal of water; (g) cooling the reaction vessel 101; and (h) releasing the vacuum and removing an oligomeric composition from the system 100.

This summary is not intended to identify all the essential features of the claimed subject matter, nor is it intended to use in determining or limiting the scope of the claimed subject matter.

### BRIEF DESCRIPTION OF DRAWINGS

The detailed description of drawings is outlined with reference to the accompanying figures. In the figures, the left-most digit(s) of a reference number identifies the Figure in which the reference number first appears. The same numbers are used throughout the drawings to refer like features and components.

FIG. 1 demonstrates a system/system assembly 100 for carrying out oligomerization of α-hydroxy acids, wherein said system/system assembly 100 comprises: a reaction vessel 101, distillation column 102, inlet valve 103, reflux divider 104, condenser 105, vacuum pump means 106 to adjust and regulate the vacuum within the system 100, distillate collector 107, a vessel 108 for feeding α-hydroxy acids, peristaltic pump 109, stirring means 110 for stirring the reaction vessel 101, heating means 111 for heating the reaction vessel 101, means 114 to adjust and regulate the heating means 111 for heating the reaction vessel 101, means 112 to measure the temperature of the reaction vessel 101, and means 113 for regulating or maintaining the temperature of the distillation column 102.

FIG. 2 demonstrates the progression in molecular weights of lactic acid oligomers with reaction time when oligomerization reaction is performed without portion-wise addition of lactic acid

FIG. 3(A) demonstrates the progression in molecular weights of lactic acid oligomers with reaction time when oligomerization reaction is performed with portion-wise addition of lactic acid

FIG. 3(B) illustrates the progressive reduction in lactic acid remaining in the reaction vessel during oligomerization reaction performed with portion-wise addition of lactic acid

### DETAILED DESCRIPTION

Reference throughout the specification to “various embodiments,” “some embodiments,” “one embodiment,” “alternate embodiment”, or “an embodiment” means that a particular feature, structure, or characteristic described in connection with the embodiment is included in at least one embodiment. Thus, appearances of the phrases “in various embodiments,” “in some embodiments,” “in one embodiment,” or “in an embodiment”, “in an alternate embodiment”, or “in a related embodiment” in places throughout the specification are not necessarily all referring to the same embodiment. Furthermore, the particular features, structures or characteristics may be combined in any suitable manner in one or more embodiments.

Reference throughout the specification to “system”, “system assembly”, “present system”, or “instant system” means the same system. Further reference throughout the specification to “components”, “component”, “features”, or “feature” means a constituent or group of constituents embodying the system.

Before the present apparatus and process is described, it is to be understood that this disclosure is not limited to the particular apparatus and process as described, as there can be multiple possible embodiments which are not expressly illustrated in the present disclosure but may still be practicable within the scope of the present disclosure.

Also, the technical solutions offered by the present disclosure are clearly and completely described below. Examples in which specific conditions may not have been specified, have been conducted under conventional conditions or in a manner recommended by the manufacturer.

The present disclosure relates to the oligomerization of α-hydroxy acids (AHAs). For the purpose of the instant disclosure, oligomerization is defined as a process of converting α-hydroxy acids to their oligomeric forms; oligomers being molecules made from the condensation of a few monomeric units.

In one embodiment, the present disclosure relates to oligomerization of a α-hydroxy acids, wherein the boiling point of the α-hydroxy acid ranges from 90 °C to 200 °C; and particularly from 100 °C to 150 °C.

As is known to a skilled person in the art, α-hydroxy acids, or alpha hydroxy acids (AHAs), are a class of chemical compounds that consist of a hydroxyl group on the carbon alpha to or adjacent to a carboxylic acid functionality, or simply, compounds containing a carbon atom carrying a hydroxyl group and a carboxylic acid group. α-Hydroxy acids are classically prepared by addition of hydrogen cyanide to a ketone or aldehyde, followed by acidic hydrolysis of the resulting cyanohydrin product. Prominent examples include glycolic acid, lactic acid, malic acid, tartaric acid, citric acid, and others. In a preferred embodiment, the monomeric α-alpha-hydroxy acids are selected from lactic acid and glycolic acid.

It is known that lactic acid is an organic compound produced via fermentation of carbohydrates by different microorganisms. While most lactic acid bacteria display amylase activity that is responsible for lactic acid production, they require complex nutrients and slightly lower fermentation temperatures (<45 °C) for the production.

This may sometimes lead to poor productivity due to the amylase production in the initial step, causing a long lag phase adding to increased costs and contamination risks. Certain fungi including Rhizopus sp. are also known to generate high content of lactic acid.

In one embodiment, the AHA (α-hydroxy acid) is lactic acid, its molecular formula being CH₃CH(OH)COOH. It is highly miscible in water and is produced via chemical or biological synthesis as well as isolated from natural sources. Lactic acid or its conjugate base lactate (or the lactate anion) is applied as a synthetic intermediate in many organic syntheses and in various biochemical industries.

In another embodiment, the AHA (α-hydroxy acid) is glycolic acid. As is known to a skilled person in the art, glycolic acid is hydroxyacetic acid, having chemical formula HOCH₂CO₂H. It is a colourless, odourless and hygroscopic crystalline solid, that is also highly soluble in water. Glycolic acid is widespread in nature and also extensively used in various skin-care products. Glycolate (sometimes spelled “glycollate”) is a salt or ester of glycolic acid.

As is known to a skilled artisan, glycolic acid is naturally produced by a variety of microorganisms by the oxidation of ethylene glycol and hydrolyzation of glycolonitrile. Chemolithotrophic iron- and sulphur oxidizing bacteria used in acidic biomining are also known to produce glycolic acid by partially unknown metabolic routes.

The present disclosure further relates to a system assembled for carrying out oligomerization of α-hydroxy acids.

In one embodiment, the system assembly comprises a reaction vessel. For the purpose of the instant disclosure, it is clarified that in the present disclosure, the term “reaction vessel” pertains to a vessel, reactor, container, flask, beaker, or any such apparatus that is used to contain reactants taking part in a reaction.

In a related embodiment, the reaction vessel is a flask; preferably a round bottom flask; more preferably a round bottom flask with more than one neck; and particularly a three neck round bottom flask.

In another embodiment, the system assembly comprises a distillation column. As is known to a skilled person in the art, a distillation column provides a surface for condensing and vaporizing fluids before their vapours enter a condenser. Thus, it aids the separation and consequently the concentration of the more volatile component in one fraction and the less volatile components in the other fraction.

In a related embodiment, the distillation column is applied to carry out simple, fractional, steam distillation, or reactive distillation; particularly, reactive distillation.

Reactive distillation is an intensification technique that integrates chemical reaction and separation via distillation in a single apparatus. In many separation processes, a hypothetical zone or stage referred to as a theoretical plate, may be perceived in which two phases, such as the liquid and vapor phases of a substance, establish an equilibrium with each other.

In a further embodiment, the distillation column is packed with a structured material. Packing optimizes the separation process by providing a large, wet surface where chemical separation or mass transfer, can take place. In the process of mass transfer, separation is usually achieved either through the opposing forces of heat and pressure that drives water vapor upward or gravity that impels liquid material downward. Packing amplifies these forces and facilitate faster and more efficient chemical separation processes.

Packing materials come in a variety of different forms and are chosen on the basis of their material to fit the surface area, weight, corrosion resistance and pressure drop that is necessary. Thus, for instance, metal packing materials are known for their strength, but plastic packing materials are typically more cost-effective.

Ceramic packing materials can be brittle, but they are in high demand for corrosive substances such as chemical waste because they resist corrosion well.

Some requirements that packing materials must meet to perform effectively involve chemical inertness with components to be separated, strong yet light in weight. Also crucial is the adequacy of number of passageways for fluids to flow through without obstructions or pressure drop and availability of ample surface area for contact between the fluids.

While there are two main types of packing-random and structured-structured packing is organized packing that channelises liquid material into a specific shape. It uses discs composed of materials such as metal, plastic or porcelain with their internal structures arranged into different types of honeycombed shapes that are found within cylindrical column. Structured packing cylinders are precisely engineered to provide a large surface area for the fluid to contact without causing resistance that impedes its flow. Some types of structured packing materials have additional textured designs to increase contact by liquid spreading which is particularly important in low-pressure applications where internal pressure alone cannot be relied upon to spread the liquid.

Structured packing typically has a smaller pressure drop allowing for a larger flow volume than random packing which is beneficial in separations involving extremely low pressure or extremely high flow rates. The low pressure provides other advantages such as higher volatility, which is beneficial in difficult separation processes along with increased energy efficiency and reduced foaming.

Due to their tightly organized internal infrastructure, structured packing also increases efficiency and demonstrates ability to pack more volume. Moreover, the higher capacity that is possible with structured packing, in turn, leads to increased operating rates.

Thus, in a further embodiment, the distillation column is packed with a structured material; particularly having a theoretical plate count ranging from 5 to 25. In a preferred embodiment, the distillation column comprises a structured packing having theoretical plate count ranging from 5 to 15.

In a related embodiment, the system assembly comprises of at least one valve, particularly an inlet valve. It is known to a skilled person in the art that a valve is a device or natural object that regulates, directs or controls the flow of a fluid (gases, liquids, fluidized solids, or slurries) by opening, closing, or partially obstructing various passageways. For the purpose of the instant disclosure, the type of valve is at least one of hydraulic, motor driven, manual, or pneumatic valves.

In a further embodiment, the system assembly comprises of at least one reflux divider. A reflux divider, as is widely known, is a pneumatic valve located between condenser and top of the column, that helps in maintaining the desired reflux ratio in the distillation column. This ensures that the α-hydroxyacids reflux closer to the reaction vessel and mostly only the water gets distilled out, thus reducing loss of the corresponding α-hydroxyacids in the distillate. For the purpose of the instant disclosure, the reflux divider is at least one of manual reflux divider or magnetic reflux divider; preferably, a magnetic reflux divider.

In another embodiment, the system assembly comprises a condenser which is known to be a laboratory apparatus that functions by lowering the temperature of vapours in order to condense them; that is, turning them into liquids by the virtue of cooling them down.

In yet another embodiment, the system assembly comprises of a vacuum pump, conventionally applied to reduce the pressure of a system.

In a related embodiment, the system assembly further comprises a distillate collector, which is typically used for collecting the distillate obtained during the distillation process. As described in the foregoing paragraphs, for the purpose of the instant disclosure, it is clarified that, the term “collector” pertains to multi necked flasks, vessels, containers, or any such apparatus configured with multiple necks, that is used to contain the distillate obtained during the oligomerization process.

The instant disclosure discloses that in one embodiment, the reaction vessel is configured to connect to a distillation column, preferably, at the proximal end of a distillation column.

In another embodiment, the distillation column is further configured to connect to a reflux divider; preferably, at its distal end; and more preferably, the distal end of a distillation column is configured to connect to the proximal end of a reflux divider.

In yet another embodiment, the reflux divider is configured to connect to a condenser and a distillate collector; preferably, the distal end of a reflux divider is configured to connect to a condenser; more preferably, the distal end of a reflux divider is configured to connect to the proximal end of a condenser, while being provided with means for connecting to the distillate collector.

In a further embodiment, the condenser, and the distillate collector are configured to connect to a vacuum pump; particularly, the distal end of a condenser is configured to connect to a vacuum pump, while one of the necks of the distillate collector is connected to the vacuum pump.

The instant disclosure further discloses that in one embodiment, the distillation column is configured to receive α-hydroxy acids via an inlet valve, particularly, an inlet valve close to its distal end.

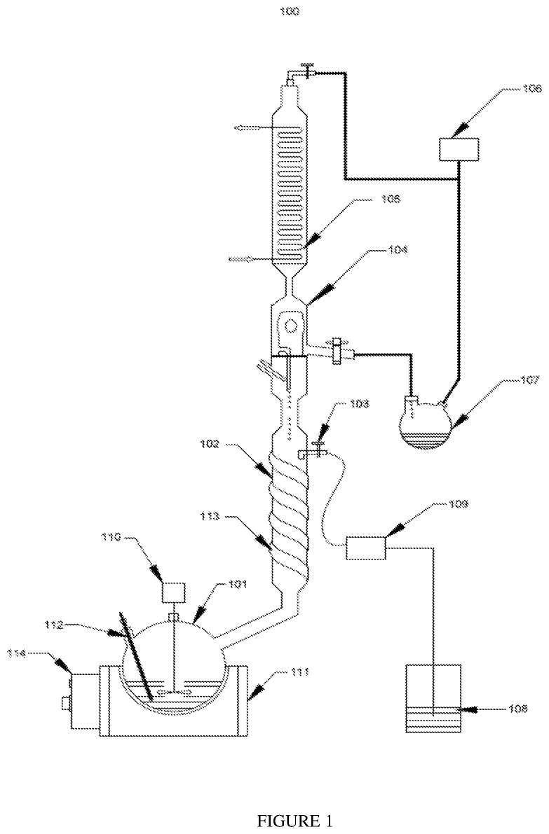

Now referring to the FIG. 1 of the instant disclosure, in one embodiment, the α-hydroxy acids are fed portion-wise or continually to the reaction vessel 101 via the inlet valve 103 positioned close to the distal end of the distillation column 102 in a controlled mode. Controlled mode or manner of addition of the α-hydroxy acids is achieved by using means 109 for regulated addition such as a peristaltic pump or any other dosing pump. Further, as the entire system itself operates under vacuum or reduced pressure, controlled opening of a valve also drives the controlled addition of the α-hydroxy acids.

For the purpose of this disclosure, controlled mode may include batch mode, fed-batch mode, continuous mode, semi-continuous mode, or any combination thereof.

Furthermore according to FIG. 1, an exemplary embodiment of the present disclosure relates to a system 100 assembled for carrying out oligomerization of α-hydroxy acids, the said system assembly 100 comprising: a reaction vessel 101 charged with a portion of the total α-hydroxy acids configured to connect to proximal end of a distillation column 102; the distillation column 102 configured to receive the α-hydroxy acids via an inlet valve 103 close to its distal end; the inlet valve 103 for feeding the remaining α-hydroxy acids in a controlled manner configured to connect to a vessel 108; the distal end of the distillation column 102 further configured to connect to proximal end of a reflux divider 104; the distal end of the reflux divider 104 configured to connect to proximal end of a condenser 105, while being provided with means for connecting to the distillate collector 107; distal end of the condenser 105 and the distillate collector 107 being provided with means for connecting to a vacuum pump 106; characterized in that, the α-hydroxy acids are fed portion-wise or continually to the reaction vessel 101 via the inlet valve 103.

In one embodiment, the system assembly further comprises stirring means for stirring the reaction vessel.

For the purpose of the instant disclosure, “stirring means” may relate to a device or an apparatus for carrying out the agitation, shaking or mixing, of components. In a preferred embodiment, the stirring means is at least one of electric, overhead, magnetic, manual, or mechanical stirrer; particularly, an anchor type overhead stirrer.

In another embodiment, the system assembly further comprises heating means for heating the reaction vessel.

For the purpose of the instant disclosure, the term “heating means” relates to a device or an apparatus for raising or maintaining the temperature of the reactants or reactive components. In a preferred embodiment, the heating means is at least one of heating mantle, steam heaters, or oil heaters; particularly, a heating mantle.

In yet another embodiment, the system assembly further comprises means for adjusting, measuring or regulating the temperature of the reaction vessel.

For the purpose of the instant disclosure, the “means to measure the temperature of the reaction vessel” relates to at least one of digital thermometer, thermocouple coupled with a thermostat, or liquid bulb capillary thermometer; particularly, a thermocouple coupled with a thermostat.

As is known to a skilled person in the art, a thermostat is a device that maintains a system at a constant temperature. It often consists of a bimetallic strip that bends as it expands and contracts with temperature, thus breaking and making contact with an electrical power supply.

In a preferred embodiment, said means to measure temperature of the reaction vessel is inserted in a thermo-pocket of the reaction vessel; more preferably, the thermocouple is inserted in a thermo-pocket of the reaction vessel.

In one embodiment, the system assembly further comprises means to adjust or regulate vacuum within the system.

In a preferred embodiment, “the means to adjust or regulate vacuum within the system” is a vacuum pump, as is disclosed in the above paragraphs.

In another embodiment, the system assembly further comprises means for heating, adjusting, regulating or maintaining temperature of the distillation column. In a preferred embodiment, the means for heating, adjusting, regulating or maintaining temperature of the distillation column is a heating tape, particularly comprising of hot water or oil circulation.

In yet another embodiment, “the means for connecting to the distillate collector” is an outlet valve.

In another embodiment, “means to regulate and adjust the heating means for heating the reaction vessel” is a rheostat, conventionally referred to as regulator, lever, controller, button, or knob.

Further, in a related embodiment, the system assembly also comprises means to adjust or regulate flow of the α-hydroxy acids; particularly during the process of charging the reaction vessel. In a preferred embodiment, the means to adjust or regulate flow of the α-hydroxy acids is a peristatic pump.

Now referring to the FIG. 1, an exemplary embodiment of the present disclosure relates to a system, as described in the foregoing paragraphs, wherein the system 100 further comprises:
stirring means 110 for stirring the reaction vessel 101;
heating means 111 for heating the reaction vessel 101;
means 112 to measure temperature of the reaction vessel 101;
means 106 to adjust or regulate vacuum within the system 100;
means 109 to adjust or regulate flow of the α-hydroxy acids; and
means 113 for regulating or maintaining temperature of the distillation column 102.

The present disclosure also relates to a process for carrying out oligomerization of α-hydroxy acids. As is known to a skilled person in the art, the condensation of α-hydroxy acids to its oligomeric forms takes place by removal of water, in one or a plurality of steps, at atmospheric pressure or under reduced pressure, until an oligomeric composition is obtained having an average degree of polymerization (DP).

In one embodiment, the process comprises charging the reaction vessel with a portion of the total α-hydroxy acids.

In another embodiment, the process comprises reducing and further maintaining reduced pressure of said system.

In yet another embodiment, the process comprises heating and maintaining the temperature of the reaction vessel to enable removal of water from the α-hydroxy acids.

In a related embodiment, the process comprises increasing and further maintaining the increased temperature of distillation column to enable evaporation of water.

In one embodiment, the process comprises feeding of the remaining α-hydroxy acids to the reaction vessel for further oligomerization.

In another embodiment, the process comprises cooling the reaction vessel.

In further embodiment, the process comprises releasing the vacuum and removing said oligomeric composition from the system.

FIG. 1 displays a further exemplary embodiment of the present disclosure and relates to a process for carrying out oligomerization of α-hydroxy acids by the system 100 described vide supra; said process comprising:
a) charging the reaction vessel 101 with a portion of the total α-hydroxy acids;
b) reducing and further maintaining reduced pressure of said system 100;
c) heating reaction vessel 101 to reflux temperature to enable oligomerisation of the α-hydroxy acids;
d) maintaining temperature of the distillation column 102 to enable evaporation of water;
e) feeding the remaining α-hydroxy acids to the reaction vessel 101 via inlet valve 103 for further oligomerization;
f) maintaining the reflux temperature of reaction vessel 101 to effect oligomerization of the α-hydroxy acids with simultaneous removal of water;
g) cooling the reaction vessel 101; and
h) releasing the vacuum and removing an oligomeric composition from the system 100.

In a related embodiment, temperature for oligomerization varies from 140 °C to 220 °C; particularly from 160 °C to 200 °C.

In a yet another related embodiment, temperature of the distillation column 102 is maintained between 30 °C to 120 °C; particularly between 50 °C to 100 °C.

In one embodiment, the vacuum of the system is maintained between of 100 mbar to 760 mbar; particularly, between of 300 mbar to 600 mbar.

In another embodiment, heating of the reaction vessel is continued after charging the α-hydroxy acids to the reaction vessel for a period of 40 minutes to 200 minutes; particularly, for a period of 60 minutes to 180 minutes.

The instant disclosure further discloses that in one embodiment, a portion of the α-hydroxy acids varies from 1% w/w to 99% of the total α-hydroxy acids; particularly, from 10% w/w to 90% w/w.

In another embodiment, the remaining α-hydroxy acids varies from 40% to 90%; particularly from 60% to 80%.

In further embodiment, the present disclosure discloses that the rate of charging of the reaction vessel with remaining α-hydroxy acids is less than 10% w/w of reaction mass per minute; particularly, less than 5% w/w of reaction mass per minute.

In one embodiment, the α-hydroxy acids that get collected in the distillate collector are less than 5% w/w of the total input of the α-hydroxy acids; and particularly, less than 2% w/w of the total.

The present disclosure discloses an oligomerization system and process that synergistically helps in maximising the yields of the oligomeric composition and minimizing the wastage of the starting α-hydroxy acids.

In one embodiment, the oligomerization process is completed within 10 hours; and particularly, within 5 hours.

The present disclosure discloses an oligomerization system and process that synergistically help in decreasing the time required for the oligomerization, and thus, decreasing the overall working time of the system.

The present disclosure also discloses that in one embodiment, molecular weight of the oligomers ranges from 400 Da to 2700 Da; preferably range between 600 Da to 2500 Da and more preferably between 900-1200 Da.

In another embodiment, the oligomeric composition comprises oligomers having monomeric units between 4 to 20; preferably, between 5 to 15.

In yet another embodiment, oligomers produced are α-hydroxy acid oligomers; particularly, lactic acid oligomers or glycolic acid oligomers or mixed oligomers.

In one embodiment, the oligomeric composition comprises of lactic acid oligomers.

Now referring to the FIG. 1, an exemplary embodiment of the present disclosure relates to a system 100 assembled for carrying out oligomerization of lactic acid, the said system assembly 100 comprising: a reaction vessel 101 charged with a portion of the total α-hydroxy acids configured to connect to proximal end of a distillation column 102; the distillation column 102 configured to receive lactic acid via an inlet valve 103 close to its distal end; the inlet valve 103 for feeding the remaining α-hydroxy acids in a controlled manner configured to connect to a vessel 108; the distal end of the distillation column 102 further configured to connect to proximal end of a reflux divider 104; the distal end of the reflux divider 104 configured to connect to proximal end of a condenser 105, while being provided with means for connecting to the distillate collector 107; distal end of the condenser 105 and the distillate collector 107 being provided with means for connecting to a vacuum pump 106; characterized in that, lactic acid is fed portion-wise or continually to the reaction vessel 101 via the inlet valve 103.

Further referring to the FIG. 1, an embodiment of the present disclosure relates to a system as described in the foregoing paragraphs, wherein the system 100 further comprises:
stirring means 110 for stirring the reaction vessel 101;
heating means 111 for heating the reaction vessel 101;
means 112 to measure temperature of the reaction vessel 101;
means 106 to adjust or regulate vacuum within the system 100;
means 109 to adjust or regulate flow of the lactic acid; and
means 113 for regulating or maintaining temperature of the distillation column 102.

Additionally, referring to FIG. 1, an exemplary embodiment of the present disclosure relates to a process for carrying out oligomerization of lactic acid in the system 100 described earlier; said process comprising:
a) charging the reaction vessel 101 with a portion of the total lactic acid;
b) reducing and further maintaining reduced pressure of said system 100;
c) heating reaction vessel 101 to reflux temperature to enable oligomerisation of the lactic acid;
d) maintaining temperature of the distillation column 102 to enable evaporation of water;
e) feeding the remaining lactic acid to the reaction vessel 101 for further oligomerization;
f) maintaining the reflux temperature of reaction vessel 101 to effect oligomerization of lactic acid with simultaneous removal of water;
g) cooling the reaction vessel 101; and
h) releasing the vacuum and removing an oligomeric composition of lactic acid from the system 100.

A further exemplary embodiment of the present disclosure relates to a system and process for carrying out oligomerization of lactic acid, said process comprising charging a three neck round bottom flask with a portion of the total solution of lactic acid; attaching a thermocouple to one side neck for temperature measurement, attaching a distillation column packed with steel gauze to the other side neck, and connecting an overhead stirrer to the central neck for continuous stirring, such that the temperature of packed column is maintained at about 55-60 °C to avoid loss of lactic acid; attaching a reflux divider to the column to maintain a desirable reflux ratio, and attaching a water condenser at the top of the column to avoid loss of lactic acid; maintaining the system at a vacuum pressure of about 500 mbar throughout the process; stirring at about 400 rpm; gradually increasing the temperature of the rection vessel to about 40-50 °C, and distilling out free water; then increasing the temperature to about 170 °C, and further maintaining for the first one hour; followed by feeding remaining portion of lactic acid at a rate of about 0.78-1.0 L/h using a peristaltic pump through a feeding valve fitted near the top of column; and maintaining the vessel at about 170 °C with continuous stirring for about 3 hours to attain the desired molecular weight and composition of the oligomer, such that lactic acid addition is in portions; the system being maintained such that only lactic acid flows into the stirred tank reactor while free water evaporates and gets collected as distillate. The heated column with structured packing helps to separate the lactic acid and water vapors which reduces loss of lactic acid in the distillate.

In a related embodiment, the molecular weight of the lactic acid oligomers ranges from 900-1500 Dalton.

In a preferred embodiment, the oligomeric composition comprises lactic acid oligomers; particularly, wherein, the molecular weight of the lactic acid oligomers ranges from 900-1200 Dalton.

In another embodiment, the oligomeric composition comprises of glycolic acid oligomers.

Furthermore, in one embodiment, the process disclosed hereinabove is a batch process.

Alternatively, in another embodiment, the process disclosed hereinabove is a continuous process.

Further, alternatively, in yet another embodiment, the process disclosed hereinabove is a fed-batch process.

Various modifications to the embodiment will be readily apparent to those skilled in the art and the generic principles herein may be applied to other embodiments. However, one of ordinary skill in the art will readily recognize that the present disclosure is not intended to be limited to the embodiments illustrated but is to be accorded the widest scope consistent with the principles and features described herein.

The foregoing description shall be interpreted as illustrative and not in any limiting sense. A person of ordinary skill in the art would understand that certain modifications could come within the scope of this disclosure.

The features and properties of the present disclosure are described in further detail below with reference to examples.

### Example 1

Oligomerization Process without Portion-Wise Addition of Lactic Acid

For the present experiment, approximately 300 grams of lactic acid (about 87% purity by assay and about 63% by HPLC) was taken in a three neck round bottom flask. A thermocouple was attached to one side neck for temperature measurement, a distillation column (diameter—~25 mm, height—~0.5 ft.) packed with steel gauge was attached to another neck, and an overhead stirrer was connected to the central neck for continuous stirring.

Further, a reflux divider was attached to the column to maintain the reflux ratio, and a water condenser was attached on the top of the column to avoid the loss of water or lactic acid. Vacuum of the system was maintained at about 500 mbar throughout with a vacuum controller. Stirring was maintained at about 400 rpm with overhead stirrer. Temperature of the reaction vessel was gradually increased. Water distilled out at about 40-50 °C, after which, temperature was raised up to 170 °C, and maintained at about 170 °C for about 3 hours. The distillate was collected during the process. The following results were achieved by the end of the process (see Table 1):

TABLE 1

| Time required for oligomerization | About 7 hours |
|---|---|
| % Lactic acid in the obtained oligomer | About <2% |
| Molecular weight of the oligomer | About 900-1200 Da |
| Concentration of Lactic acid in the distillate | 6-8% |

In the present study, the oligomerization process performed without continuous addition of lactic acid in about 7 h resulted in oligomers of lactic acid having MW from 900-1200 Da with remaining distillate concentration of 6-8% of lactic acid.

### Example 2

Study of the Oligomerization Process without Portion-Wise Addition of Lactic Acid: Study of Progression of Oligomer Molecular Weight with Reaction Time

For carrying out the study of molecular weight development with time in an oligomerization process without continuous addition of lactic acid, the experiment described in Example 1 was repeated, and the following was determined.

Table 2 includes comprehensive information on the development of molecular weight with time during an oligomerization process (without continuous addition of lactic acid)

TABLE 2

| Reaction time | Oligomers | Approximate lactic acid remaining |
|---|---|---|
| (minutes) | MW (Da) | in reaction vessel (%) |
| 87 | 270 | 53.23 |
| 151 | 300 | 38.74 |
| 210 | 456.48 | 15.52 |
| 270 | 799 | 2 |
| 330 | 1247 | 0.89 |
| 390 | 1905 | 0.41 |
| 450 | 2151 | 0.18 |

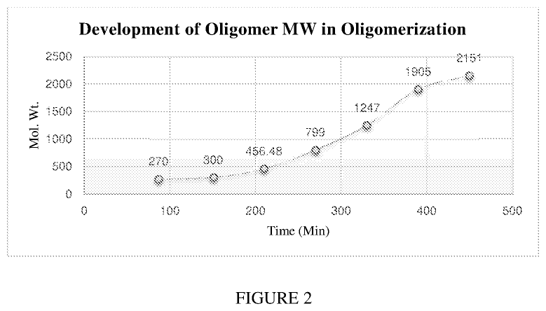

As seen from FIG. 2, the above process yields lactic acid oligomers having MW of approximately 2151 Da in about 450 minutes, with an approximate 0.18% lactic acid left over in the reaction flask.

### Example 3

Oligomerization Process with a Portion-Wise Addition of Lactic Acid

For the present experiment, a three neck round bottom flask was charged with approximately 300 g of lactic acid (about 87% purity by assay and about 63% by HPLC).

A thermocouple was attached to one side neck for temperature measurement, a distillation column (diameter—~25 mm, height—~0.5 ft.) packed with structured steel gauze packing was attached to the neck on the other side, and an overhead stirrer was connected to the central neck for continuous stirring. The temperature of packed column was maintained at about 60 °C to avoid loss of lactic acid.

Further, a reflux divider was attached to the column to maintain the reflux ratio, and a water condenser was attached at the top of the column to avoid the loss of water or lactic acid. Vacuum of the system was maintained at about 500 mbar throughout the process. Stirring was maintained at about 400 rpm with overhead stirrer. The temperature of the reaction flask was gradually increased. Then, at about 40-50 °C temperature, free water was distilled out.

After about 35 minutes, temperature of the reaction flask was increased to about 170 °C. For first one-hour, the temperature was maintained at about 170 °C.

After maintaining the temperature for about 1-hour, feeding of remaining portion of lactic acid (about 1000 g) was initiated through a feeding valve fitted near the top of column at a flowrate about 15 mL/min (between 0.780-1 L/h). This flow rate along with the maintained column temperature helps to avoid loss of lactic acid and maintains the temperature of the stirred reaction flask. This helps to complete oligomerization in 5 h. A higher flow rate decreases reaction temperature in the flask, at the bottom of the system, leading to extended oligomerization time, while a lower flow rate leads to loss of lactic acid in the distillate. It was noted that during the feeding of lactic acid, mostly free water was distilled out without loss of lactic acid. A skilled artisan may adjust the flow rate of addition of the remaining portion of lactic acid and further also vary the temperature of the structured column such that free monomeric lactic acid refluxes towards the bottom of the system and does not get distilled out from the top of the column.

After complete feeding, the reaction vessel was maintained at about 170 °C with continuous stirring for about 3 hours to attain the desired molecular weight and composition of the oligomer.

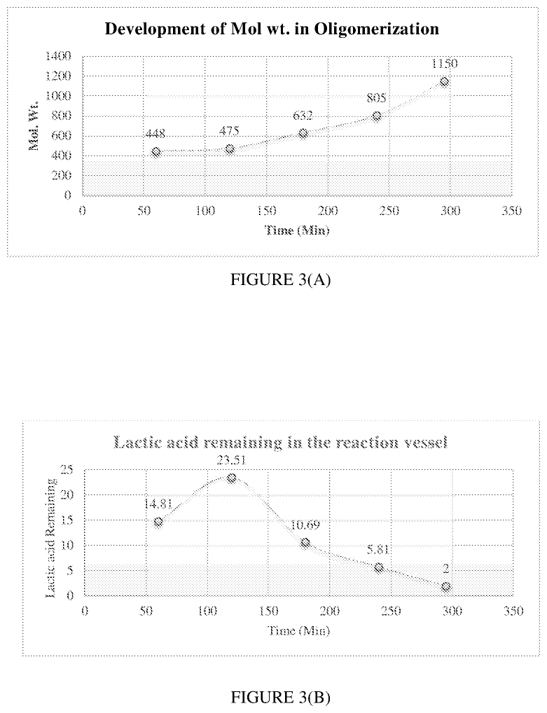

As illustrated in FIGS. 3(A) and 3(B) desired oligomers were obtained with molecular weight of about 900-1200 Da having less than 2% lactic acid. Further, it was observed that the loss of lactic acid in the distillate was only about 2-5% w/w of the input/starting lactic acid.

Table 3 shows the approximate/average results of two batches of oligomerization involving portion-wise addition of lactic acid.

TABLE 3
Properties of lactic acid oligomers

| Condition | | | | | | | |
|---|---|---|---|---|---|---|---|
| | Feed | Portion- | | | | | |
| Temperature | added to | wise | Distillate | | Oligomer | | |
| of reaction | reaction | Feeding | Wt | % | MW | Yield | % |
| vessel (°C) | vessel (g) | (g) | (g) | LA | (Dalton) | (%) | LA |
| 170 | 302 | 1018 | 320 | 2.02 | 1164 | 78 | 1.14 |

### Example 4

Study of the Oligomerization Process with Portion-Wise Addition of Lactic Acid: Tracing the Progression/Formation of Oligomer Molecular Weight with Reaction Time

For carrying out the study of molecular weight development with time in an oligomerization process with continuous addition of lactic acid, the experiment described in Example 3 was repeated, and the following was determined.

Table 4 gives a comprehensive information on the development of molecular weight with time during an oligomerization process with portion-wise addition of lactic acid

TABLE 4
Properties of lactic acid oligomers

| | | Lactic acid | |
|---|---|---|---|
| Reaction Time | Oligomers | remaining in | Reacted Lactic |
| (min) | MW (Da) | reaction vessel (%) | acid (moles) |
| 60 | 448 | 14.81 | 2.47 |
| 120 | 475 | 23.51 | 7.88 |
| 180 | 632 | 10.69 | 2.16 |
| 240 | 805 | 5.81 | 0.236 |
| 295 | 1150 | 2.0 | 0.013 |

*Monomer feeding was initiated at about 68 minutes

### Example 5

### Oligomerization Process for Glycolic Acid

For the present experiment, a three neck round bottom flask was charged with approximately 300 g of glycolic acid (70% purity by assay).

A thermocouple- was attached to one neck for temperature measurement, a distillation column (diameter~50 mm, height~800 mm) structured column packed with steel gauze was attached to the neck on the other side, an overhead stirrer was connected to the central neck for continuous stirring. The temperature of packed column was maintained at about 60 °C to avoid the loss of glycolic acid. Further, a reflux divider was attached to the column to maintain the reflux, and a water condenser was attached at the top of the column to avoid the loss of water or glycolic acid. Vacuum of the system was maintained at about 500 mbar throughout the process. Stirring was maintained at about 400 rpm with overhead stirrer. The temperature was gradually increased.

After 35 min, temperature reached about 170 °C. For first one-hour, the temperature was maintained at about 170 °C.

After maintaining the temperature for about 1-hour, continuous feeding of glycolic acid (1000 g) was initiated through a feeding valve fitted near the top of column at a flowrate about 15 mL/min. It was noted that during the continuous feeding of glycolic acid, free water was distilled out without loss of glycolic acid.

After complete feeding, the reaction vessel was maintained at about 170 °C with continuous stirring for about 5 hours to attain the desired molecular weight and composition of the oligomer.

Desired oligomers were obtained with molecular weight of about 900-1200 Da at a yield of about 70-85%.

Table 5 shows the result of oligomerization of glycolic acid involving its portion-wise addition.

TABLE 5
Properties of obtained glycolic acid oligomers

| | Conditions | | | | |
|---|---|---|---|---|---|
| | | Feed added to | Portion- | Oligomers | |
| | Temperature | reaction vessel | wise Feed | MW* | Yield |
| | (°C) | (g) | (g) | (Da) | (%) |
| Batch | 170 | 300 | 1018 | 1010 | 76 |

*Molecular weight of oligomers of glycolic acid was determined by H¹ NMR.

The embodiments, examples and alternatives of the preceding paragraphs or the description and drawings, including any of their various aspects or respective individual features, may be taken independently or in any combination. Features described in connection with one embodiment are applicable to all embodiments, unless such features are incompatible.

The preferred embodiments of the present invention are described in detail above. It should be understood that ordinary technologies in the field can make many modifications and changes according to the concept of the present invention without creative work. Therefore, all technical solutions that can be obtained by those skilled in the art through logical analysis, reasoning or limited experiments based on the concept of the present invention on the basis of the prior art should fall within the protection scope determined by the claims.

1. A system assembled for carrying out oligomerization of α-hydroxy acids, the said system assembly comprising:
a reaction vessel charged with a portion of the total α-hydroxy acids configured to connect to proximal end of a distillation column;
the distillation column configured to receive α-hydroxy acids via an inlet valve close to its distal end;
the inlet valve for feeding the remaining α-hydroxy acids in a controlled manner configured to connect to a vessel;
the distal end of the distillation column further configured to connect to proximal end of a reflux divider;
the distal end of the reflux divider configured to connect to proximal end of a condenser, while being provided with means for connecting to the distillate collector;
distal end of the condenser and the distillate collector being provided with means for connecting to a vacuum pump;
characterized in that,
the α-hydroxy acids are fed portion-wise to the reaction vessel via the inlet valve.

2. The system of claim 1, wherein the α-hydroxy acids are fed portion-wise or continually to the reaction vessel via the inlet valve positioned close to the distal end of the distillation column in a controlled mode.

3. The system of claim 1, wherein the system further comprises:
stirring means for stirring the reaction vessel;
heating means for heating the reaction vessel;
means to measure temperature of the reaction vessel;
means to adjust or regulate vacuum within the system;
means to adjust or regulate flow of the α-hydroxy acids; and
means for regulating or maintaining the temperature of the distillation column.

4. A process for carrying out oligomerization of α-hydroxy acids by the system of claim 1; said process comprising:
a) charging reaction vessel with a portion of the total α-hydroxy acids;
b) reducing and further maintaining reduced pressure of said system;
c) heating reaction vessel to reflux temperature to enable oligomerization of the α-hydroxy acids;
d) maintaining temperature of the distillation column to enable evaporation of water;
e) feeding the remaining α-hydroxy acids to the reaction vessel via inlet valve for further oligomerization;
f) maintaining the reflux temperature of reaction vessel to effect oligomerization of α-hydroxy acids with simultaneous removal of water;
g) cooling the reaction vessel; and
h) releasing the vacuum and removing an oligomeric composition from the system.

5. The process of claim 4, wherein reflux temperature for oligomerization varies from 160 °C to 200 °C.

6. The process of claim 4, wherein temperature of the distillation column is maintained between 50 °C to 100 °C.

7. The process of claim 4, wherein the distillation column comprises a packing having theoretical plate count ranging from 5 to 15.

8. The process of claim 4, wherein the vacuum of the system is maintained between of 300 mbar to 600 mbar.

9. The process of claim 4, wherein heating of the reaction vessel is continued for a period of 60 minutes to 180 minutes after charging the reaction vessel with the total α-hydroxy acids.

10. The process of claim 4, wherein a portion of the total α-hydroxy acids varies from 10% w/w to 90% w/w of the total α-hydroxy acids.

11. The process of claim 4, wherein the boiling point of the α-hydroxy acid ranges from 100 °C to 150 °C.

12. The process of claim 4, wherein the remaining α-hydroxy acids varies from 60% to 80%.

13. The process of claim 4, wherein the rate of charging of the reaction vessel with remaining α-hydroxy acids is less than 5% w/w of reaction mass per minute.

14. The process of claim 4, wherein the α-hydroxy acids that get collected in the distillate collector are less than 2% w/w of the total input of the α-hydroxy acid.

15. The process of claim 4, wherein the oligomerization process is completed within 5 hours.

16. The process of claim 4, wherein molecular weight of the oligomers range from 600 Da to 2500 Da.

17. The process of claim 4, wherein the monomeric α-alpha-hydroxy acids are selected from lactic acid, and glycolic acid.

18. The process of claim 17, wherein the α-hydroxy acid is lactic acid.

19. The process of claim 17, wherein the α-hydroxy acid is glycolic acid.

20. The process of claim 4, wherein the oligomeric composition comprises oligomers having monomeric units between 5 to 15.

21-23. (canceled)
